(19) United States
(12) Patent Application Publication
(10) Pub. No.: US 2021/0187290 A1
(43) Pub. Date: Jun. 24, 2021

(54) SYSTEM FOR PROVIDING STIMULATION

(71) Applicant: ONWARD MEDICAL B.V., Eindhoven (NL)

(72) Inventors: Miroslav CABAN, Renens (CH); Joachim v. ZITZEWITZ, Bussigny (CH); Vincent DELATTRE, Eindhoven (NL)

(73) Assignee: ONWARD MEDICAL B.V., Eindhoven (NL)

(21) Appl. No.: 17/125,859

(22) Filed: Dec. 17, 2020

(30) Foreign Application Priority Data
Dec. 18, 2019 (EP) 19217281.5

(51) Int. Cl. A61N 1/36; A61H 1/02; A61H 23/00; A61H 7/00; A61H 15/02

(52) U.S. Cl. CPC A61N 1/36003; A61H 1/02; A61H 23/00; A61H 7/00; A61H 2201/165; A61H 15/02; A61N 1/36062; A61H 2201/10; A61H 2201/164; A61N 1/36103

(57) ABSTRACT

Systems and methods are provided for delivering neurostimulation. A system can include at least one stimulation element, the stimulation element being configured to provide stimulation to a least one tendon and/or muscle of a patient.

### CROSS-REFERENCE TO RELATED APPLICATIONS

This application claims the benefit of priority under 35 U.S.C. 119 to European Patent Application No. 19217281.5, filed Dec. 18, 2019. This application is incorporated herein by reference in its entirety.

### SUMMARY

The disclosed systems and methods relate to provision of neurostimulation, and more particularly to provision of stimulation to at least one of muscles or tendons. It is to be understood that the following detailed description is exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in and constitute a part of this specification, illustrate several embodiments consistent with the disclosure and, together with the description, serve to explain the principles of the disclosure. In the drawings:

FIG. 1 depicts exemplary promotion of peripheral neural activity in a patient, in accordance with conventional practice.

FIGS. 2 to 4 depict exemplary systems for providing neurostimulation, in accordance with disclosed embodiments.

FIG. 5 depicts an additional exemplary promotion of peripheral neural activity in a patient, in accordance with disclosed embodiments.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. For example, unless otherwise indicated, method steps disclosed in the figures can be rearranged, combined, or divided without departing from the envisioned embodiments. Similarly, additional steps may be added or steps may be removed without departing from the envisioned embodiments. Thus, the materials, methods, and examples are illustrative only and are not intended to be necessarily limiting.

Spinal cord injury (SCI) can interrupt the communication between the spinal cord and supraspinal centers, depriving these sensorimotor circuits from the excitatory and modulatory drives necessary to produce movement. After SCI, patients undergo intense rehabilitation in order to regain motor, autonomic and/or sensory function. Promotion of peripheral neural activity is standard practice in rehabilitation of SCI patients.

Typically, the promotion is done by at least one therapist, such as a physiotherapist, that presses against a tendon and/or a muscle of a patient with reduced control (such as motor control) to evoke a specific action (such as motor action, e.g., during a rehabilitation task). In other words, the promotion is typically done manually. However, delivering tendon and/or muscle stretches during the execution of a rehabilitation task is time-consuming and further often requires more than one therapist. It further requires a significant physical effort and good time coordination and must thus be performed by someone with enough training and skill. Frequently, at least one further therapist is required assuring patient safety and motivation.

Alternatively, the promotion may be done by epidural electrical stimulation (EES). EES is typically delivered by an active implantable medical device, such as an electrode array comprising at least one electrode, which is implanted into the epidural space. Consequently, patients must undergo an implantation procedure.

EES can also stimulate peripheral neural pathways. EES can recruit specific neuron populations through direct and indirect pathways. In the case of recovery of locomotion, EES applied over the lumbosacral spinal cord can activate large-diameter, afferent fibers within the posterior roots which in turn can activate motoneuron pools through synaptic connections, which in turn can activate the muscles innervated by the corresponding (peripheral) neurons. Hence, specific spinal roots can be linked to specific motor functions.

EES can increase the peripheral neural activity feeding into spinal circuits, allowing patients to perform more rehabilitation activities at a higher intensity. It is further hypothesized that the increased peripheral neural activity can promote plasticity across the lesion in the spinal cord.

The mechanism of action of EES and the tendon and/or muscle stretches are similar. Both increase the activity of peripheral neurons, namely type Ia and II sensory fibers which are part of the monosynaptic reflex pathways.

The disclosed embodiments can enable peripheral sensory fiber stimulation of a patient. In some embodiments, manual support by one or more therapists may not be required. In various embodiments, the patient need not undergo surgery for implantation of an electrode array for EES.

The disclosed embodiments can include a system for providing neurostimulation. The system can include at least one stimulation element, the stimulation element being configured to provide stimulation to a least one tendon and/or muscle of a patient.

Consistent with disclosed embodiments, stimulation may be applied from outside to the body of the patient (e.g., using a non-invasive system) and/or automatically using a stimulation element placed close to at least one tendon and/or muscle to be stimulated. In particular, this may enable time and cost-efficient optimization of stimulation, as the rehabilitation task may be performed by the patient himself without the help of another person. Further, the therapy may easily be adapted to the anatomical and/or physiological and/or pathophysiological requirements of the patient.

In particular, mechanical stimulation of the at least one tendon and/or muscle may be achieved by stretching the at least one tendon and/or muscle, for example by pushing it (forcing it to elongate) and/or by applying a vibration and/or massage and/or compression to the at least one tendon and/or muscle.

It is generally possible that tendons and/or muscles of the striated and/or smooth musculature are stimulated by the system. In other words, also inner organs may be stimulated with the system. In other words, the system may provide stimulation in order to improve motor function and/or autonomic function of a patient. Autonomic function may include sphincter function, bowel function, bladder function, ureter function, etc. The stimulation of autonomic function may be enabled by e.g., massage and/or compression of the trunk and/or parts of the trunk, such as the abdomen.

Alternatively, and/or additionally, intestinal and/or gastric glands may be stimulated by the system.

In particular, any tendon and/or muscle of a patient may be stimulated. In particular, any tendon and/or muscle of at least one arm and/or hand and/or at least one leg and/or foot of a patient may be stimulated. In particular, the at least one tendon and/or muscle may be stimulated directly and/or indirectly. In particular, stimulated muscles may include but are not limited to at least one of vastus lateralis, vastus medialis, vastus intermedius, rectus femoris, biceps femoris, semimembranosus, semitendinosus, gastrocnemius, soleus plantaris, tibialis anterior, tibialis posterior, fibularis longus, extensor digitorum longus, extensor hallucis longus, fibularis brevis, extensor hallucis longus, fibularis tertius, superior extensor retinaculum, inferior extensor retinaculum, popliteus, flexor digitorum longus, flexor digitorum brevis, flexor hallucis longus, extensor digitorum brevis, tendo calcaneus, abductor hallucis, quadratus plantae, flexor hallucis brevis, flexor digiti minimi brevis, abductor digiti minimi, deltoid, pectoralis major, biceps brachii, brachioradialis, flexor carpi radialis, flexor carpi ulnaris, extensor carpi radialis longus and/or extensor carpi radialis brevis. In particular, stimulated tendons may be any tendon related to the muscles cited above, such as the Achilles tendon and/or plantar aponeurosis.

In particular, at least one tendon and/or muscle of a leg of a patient may be stimulated to enable rehabilitation of walking, stepping, standing up, sitting down, grasping etc.

Tendons and/or muscle targeted by the system may promote a biomechanical action such as knee extension and/or flexion and/or elbow extension and/or flexion and/or ankle plantar-flexion and/or dorsi-flexion and/or hand (wrist) palmar flexion and/or dorsiflexion and/or pronation and/or supination of the forearm and/or inversion and/or eversion of the sole of the foot. In general, a biomechanical action such as abduction and/or adduction and/or elevation and/or depression and/or rotation may be promoted.

In particular, the action of a tendon may be determined by the tendon tap test.

In particular, a single tendon and/or muscle and/or a set of tendons and/or muscles, such as a pair of tendons and/or muscles associated with antagonist muscles may be stimulated by the system. In the latter case, one system may be used to promote flexion and extension of the same joint.

The system may comprise at least one fixation element to fixate the system close to the tendon and/or muscle to be stimulated. In particular, the fixation element may be or may comprise at least one of a tape, an adhesive tape and/or an adhesive element, a patch, a strap, a cuff, a belt, a clasp, a rubber band, a bandage, a garment (e.g., a sock, a stocking, tights, trousers, a shirt, a gauntlet and/or a glove) and/or any other type of fixation element. The fixation element may be at least partially made of at least one of silicone, textile, rubber, natural rubber, any type of metal and/or any kind of plastic. The fixation element may comprise elastic and/or non-elastic elements. The fixation element may comprise a closure element comprising at least one of a hook-and-loop fastener, a hook, a button, a loop and/or any other type of closure element. In particular, the fixation element may enable that the system and/or stimulation element is placed in optimal position to the tendon and/or muscle to be stimulated, without the risk of losing this optimal position.

The stimulation element may be or may comprise at least one mechanical stimulation element for stimulation of the tendon and/or muscle to be stimulated. In particular, mechanical stimulation of the tendon and/or muscle may be achieved either by directly and/or indirectly stretching a tendon and/or muscle, for example by pushing it (forcing it to elongate) and/or by applying a vibration to the tendon and/or muscle. In particular, the mechanical stimulation element may enable effects close to manual stimulation, without the requirement of therapeutic staff.

In particular, the mechanical stimulation element may be or may comprise at least one of a vibration module, a massage module, a stretching module and/or a compression module. In particular, this may enable that for each individual patient and/or each individual planned movement (including but not limited to stepping, standing up, walking, sitting down, running, swimming, grasping) and/or each individual autonomic function, the best mechanical stimulation technique (i.e. vibration, massage, stretching and/or compression) is enabled and provided by the system in order to evoke a smooth movement of the patient and/or bowel, bladder, ureter and/or sphincter function. Thus, the system comprising the mechanical stimulation element may facilitate rehabilitation and/or daily live activities.

The mechanical stimulation element may provide mechanical stimulation to the at least one tendon and/or muscle of a patient adapted to and/or synchronously with an exerted muscular activity. In particular, the system may be programmed in a temporal manner such that the correct movement is promoted by the system at the correct time during the rehabilitation and/or task execution. This may enable optimal muscular activity during rehabilitation. The rehabilitation and/or task execution may comprise any movement of daily live activities, such as walking, running, standing up, sitting down, stepping, cycling, grasping, etc.

The vibration module and/or the massage module and/or the stretching module and/or compression module may be or may comprise at least one ball and/or roll and/or block and/or clamp and/or any other element providing vibration and/or massage and/or stretching to a patient.

It is generally possible that the stimulation element is the fixation element and vice versa.

In particular at least two systems according to the present invention may be linked to each other. In particular, the linked systems may be programmed in a temporal manner such that a physiological movement based on at least two extremities (such as walking, stepping, cycling, running, swimming) is enabled.

It is possible that the system is linked to a neuromodulation system for providing EES.

In particular, the system may further comprise sensors providing feedback information.

In other words, the system may be a closed-loop system or an open-loop system.

According to the present invention, the use of a system for providing stimulation to at least one tendon and/or muscle of a patient according to any of claims 1-5 is disclosed.

In particular, the patient may be a patient with spinal cord injury (SCI).

However, it may alternatively be possible that the patient is a patient suffering from stroke, Parkinson's disease, tumors leading to partial or complete paralysis, poliomyelitis, Guillain-Barre-Syndrome, multiple sclerosis and/or cerebral palsy and/or any other disease of the central and/or peripheral nervous system causing impairment of motor function and/or autonomic function (such as bowel function and/or bladder function and/or sphincter function and/or ureter function).

According to the present invention a method is disclosed, in particular a method for providing neurostimulation, comprising at least the following steps: providing one stimulation element, and using the stimulation element for stimulation to a least one tendon and/or muscle of a patient. The method may further comprise the step of fixing the stimulation element close to the tendon and/or muscle to be stimulated. The method may further comprise the step of providing mechanical stimulation of the tendon and/or muscle to be stimulated.

In particular, the mechanical stimulation may be or may comprise at least one of a mechanical vibration stimulation, a mechanical massage stimulation, a mechanical stretching stimulation. In particular, the method may be performed by using a system configured to stimulation to a least one tendon and/or muscle of a patient (P). The system can include at least one fixation element (14, 114, 214) to fixate the system (10, 110, 210) close to the tendon and/or muscle to be stimulated. The system can include at least one mechanical stimulation element (12, 112, 212) for stimulation of the tendon and/or muscle to be stimulated. The mechanical stimulation element can include at least one of a vibration module, a massage module, a stretching module, or a compression module. The mechanical stimulation element (12, 112, 212) can be configured to provide mechanical stimulation to the at least one tendon and/or muscle of a patient adapted to and/or synchronously with an exerted muscular activity.

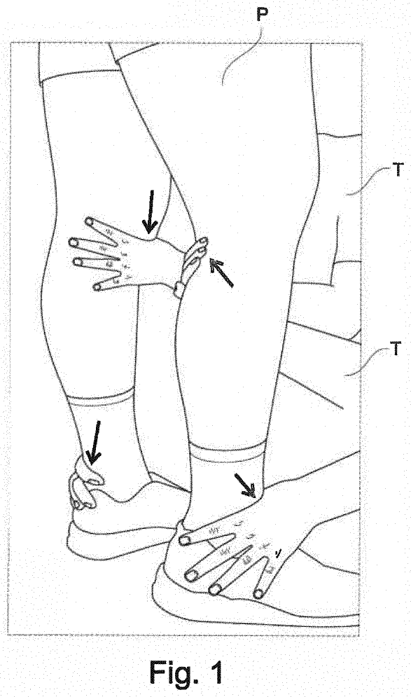

FIG. 1 shows an example of promotion of peripheral neural activity of a patient P, in accordance with conventional practice (e.g., standard practice in rehabilitation). Consistent with conventional practice, a patient P can be treated manually by two therapists T. One leg of the patient P can manually be treated by a first therapist T and the other leg can be treated by a second therapist T. The therapists T can press against tendons and/or muscles of both legs of the patient P (indicated by arrows). Not shown in FIG. 1 is that pressing against tendons and/or muscles at least partially enables peripheral neural activity of the legs of the patient P.

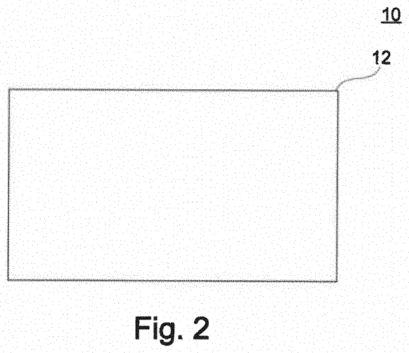

FIG. 2 shows an example of an embodiment of the system for providing neurostimulation according to the present invention with which the method according to the present invention can be performed. In this embodiment, the system 10 comprises a stimulation element 12. In an alternative embodiment, the system 10 comprises more than one stimulation element 12. It is generally possible, that the stimulation elements 12 of the system 10 are connected. In this embodiment, the stimulation element 12 is configured to provide stimulation to at least one tendon and/or muscle of a patient P. Not shown in FIG. 2 is that the stimulation element 12 can be or include, or generally be or include, a mechanical stimulation element 12 for stimulation of the tendon and/or muscle to be stimulated. Not shown in FIG. 2 is that the mechanical stimulation element 12 can be or include a massage module. In an alternative embodiment, the mechanical stimulation element 12 could be or include at least one of a vibration module, a stretching module, or a compression module. In general, the mechanical stimulation element 12 could be or could comprise at least one of a vibration module, a massage module, a stretching module, a compression module.

Consistent with disclosed embodiments, the system 10 can be used for providing stimulation to a least one tendon and/or muscle of a patient P. In some embodiments, the patient P can be a patient P with spinal cord injury (SCI). In general, the system 10 could perform a method for providing stimulation, including at least the following steps: providing one stimulation element 12, and using the stimulation element 12 for stimulation to a least one tendon and/or muscle of a patient P.

The method could further comprise the step of providing mechanical stimulation of the tendon and/or muscle to be stimulated. The mechanical stimulation could be or could comprises at least one of a mechanical vibration stimulation, a mechanical massage stimulation, a mechanical stretching stimulation, a mechanical compression module. In general, the method could be performed by using the system 10, 10, 210 according to one of FIGS. 2 to 5.

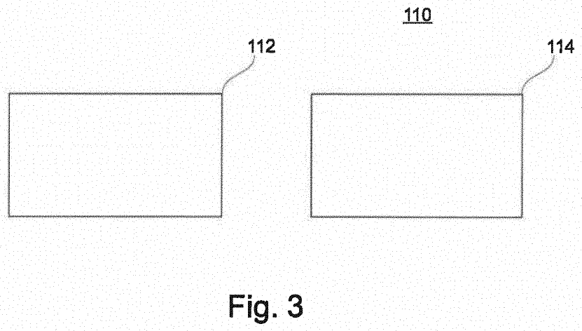

FIG. 3 depicts an exemplary system for providing neurostimulation according to the present invention with which the method according to the present invention can be performed. The system 110 can include the structural and functional features as disclosed for the system 10 disclosed in FIG. 2. The corresponding references are indicated as 100+x (e.g., stimulation element 112). In this embodiment, the system 110 further includes a fixation element 14. In this embodiment, the fixation element 114 fixates the system 110 close to the tendon and/or muscle to be stimulated. Not shown in FIG. 2 is that the fixation element 114 and the stimulation element 112 can be connected directly. In various embodiments, the fixation element 114 and the stimulation element 112 can be connected indirectly. Not shown in FIG. 3 is that the system 110 could generally include more than one fixation element 114 and/or more than one stimulation element 112.

In general, the system 110 can perform a method for providing neurostimulation, including at least the following steps: providing one stimulation element 112, and using the stimulation element 112 for stimulation to a least one tendon and/or muscle of a patient P. The method could further include the step of fixing the stimulation element 112 close to the tendon and/or muscle to be stimulated. Not shown in FIG. 3 is that the system 110 can be fixed close to the ankle and/or knee and/or wrist and/or elbow of a patient P.

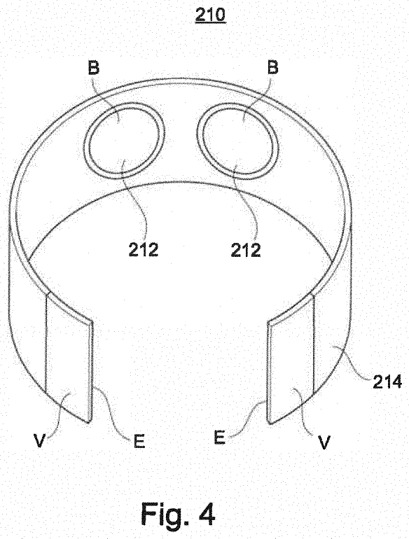

FIG. 4 shows an additional exemplary embodiment of a system for providing neurostimulation, in accordance with disclosed embodiments. The system 210 comprises the structural and functional features as disclosed for the system 110 disclosed in FIG. 3. The corresponding references are indicated as 100+x (e.g., stimulation element 212). In some embodiments, the fixation element 214 can be an elongated strap 214 provided with a hook-and-loop fastener system V at its ends E to releasably connect the ends E (where E can be an end of fixation element) of the strap 214 with each other. In various embodiments, the ends E of the strap 214 may be connected by any other type of lock, including but not limited to at least one hook, eyelet, button and/or knot.

In some embodiments, the strap 214 may be a circular strap 214. The fixation element 214 can be a circular fixation element 214. The fixation element 214 can include a fastener system V. In some embodiments, the strap 214 can be at least partially made of elastic material. In various embodiments, the strap 214 can be completely made of elastic material. In some embodiments, the strap 214 can be made of non-elastic material. In this embodiment, the strap 214 can be configured and arranged for fitting to a patient's ankle. In various embodiments, the fixation element 214 can be configured and arranged for fitting at any part of the patient's P body.

Consistent with disclosed embodiments, the stimulation element 212 can be a mechanical stimulation element 212. In particular, in some embodiments, the mechanical stimulation element 212, which can be or include a massage module, can include two balls B, (e.g., two shiatsu balls, or the like). In such embodiments, the mechanical stimulation element 212 can be movably connected with the fixation element 14. Furthermore, in such embodiments, the mechanical stimulation element 212 can be configured and arranged to provide forces to at least one tendon and/or muscle of a patient equipped with the system 210.

In some embodiments, the mechanical stimulation element 212 is configured and arranged to provide forces to the Achilles tendon of a patient P equipped with the system 210. In such embodiments, the mechanical stimulation element 212 can be configured and arranged to squeeze and/or knead the Achilles tendon of a patient P equipped with the system 210.

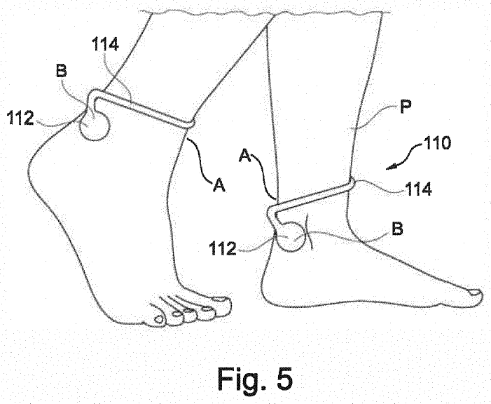

FIG. 5 shows an example of promotion of peripheral neural activity of a patient P, in accordance with disclosed embodiments. In some embodiments, a patient P is equipped with the system 110 disclosed in FIG. 3. In some embodiments, the system 110 comprises two stimulation elements 112 and two fixation elements 114. In some embodiments, each ankle A of the patient P is equipped with one stimulation element 112 and one fixation element 114. In some embodiments each stimulation element 112 comprises a massage module. Not shown in FIG. 5 is that the massage module comprises two massage balls B.

Not shown in FIG. 5 is that one massage ball B can be located at each side of the Achilles tendon of the patient P (one on the left side of the Achilles tendon and one on the right side of the Achilles tendon). Not shown in FIG. 5 is that alternatively and/or additionally, any other tendon and/or muscle and/or organ of the patient P can be stimulated with the system 110, given that the fixation element 114 places the system 110 close to the respective tendon and/or muscle and/or organ.

Consistent with disclosed embodiments, the fixation element 114 can be a U-shaped cuff. The fixation element 114 may not be closed but may be placed around the ankle. Not shown in FIG. 5 is that the fixation element 114 can be a non-elastic cuff. However, in some embodiments, the fixation element 114 may be an elastic cuff. In this embodiment, the massage balls B can squeeze against the Achilles tendon. In various embodiments, the squeeze could be at least partially generated by the cuff itself. Accordingly, in various embodiments, the fixation element 114 can be the stimulation element 112 (and vice versa). Not shown in FIG. 5 is that the system 110 can deliver sensory fiber stimulation by mechanically stimulating the Achilles tendons of the patent P. Not shown in FIG. 5 is that the system 110 can be programmed in a temporal manner such that a correct movement involving both legs of the patient P (e.g., stepping) can be promoted at the correct time during execution of a rehabilitation task. Further not shown in FIG. 5 is that the mechanical stimulation element can provide mechanical stimulation to the at least one tendon and/or muscle of a patient adapted to and/or synchronously with an exerted muscular activity.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. While certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A system for providing neurostimulation, comprising at least one stimulation element, the stimulation element being configured to provide stimulation to a least one of a tendon or a muscle of a patient.

2. The system of claim 1, wherein the system further comprises at least one fixation element to fixate the system close to the at least one of the tendon or the muscle to be stimulated.

3. The system of claim 1, wherein the stimulation element comprises at least one mechanical stimulation element configured to stimulate of the at least one of a tendon or a muscle to be stimulated.

4. The system of claim 3, wherein the mechanical stimulation element comprises at least one of a vibration module, a massage module, a stretching module, or a compression module.

5. The system of claim 4, wherein the mechanical stimulation element is configured to provide mechanical stimulation to the at least one of a tendon or a muscle of a patient, the mechanical stimulation at least one of adapted to or synchronous with an exerted muscular activity.

6. A method for providing neurostimulation, comprising:
providing stimulation to a least one of a tendon or a muscle of a patient using at least one stimulation element.

7. The method of claim 6, wherein the patient is a patient with spinal cord injury (SCI).

8. The method according to claim 6, wherein the method further comprises:
fixing the stimulation element close to the least one of a tendon or a muscle of the patient to be stimulated before providing the stimulation.

9. The method according to claim 6, wherein the provided stimulation is mechanical stimulation.

10. The method according to claim 6, wherein the provided stimulation is or comprises at least one of a mechanical vibration stimulation, a mechanical massage stimulation, a mechanical stretching stimulation.
